(19) United States
(12) Patent Application Publication
(10) Pub. No.: US 2017/0196704 A1
(43) Pub. Date: Jul. 13, 2017

(54) SURGICAL IMPACTION CENTERING APPARATUS AND METHOD

(71) Applicant: Kambiz Behzadi, (US)

(72) Inventors: Kambiz Behzadi, Pleasanton, CA (US); Paul Kardel, Greenbrae, CA (US); David Jacobs, Acton, MA (US); Jesse Rusk, Waltham, MA (US)

(73) Assignee: Kambiz Behzadi, Pleasanton, CA (US)

(21) Appl. No.: 15/235,032

(22) Filed: Aug. 11, 2016

Related U.S. Application Data
(63) Continuation-in-part of application No. 15/202,434, filed on Jul. 5, 2016.
(60) Provisional application No. 62/277,294, filed on Jan. 11, 2016.

(51) Int. Cl. A61F 2/46

(52) U.S. Cl. CPC A61F 2/4603; A61F 2/4609; A61F 2/4607; A61F 2/4612; A61F 2002/4681; A61F 2002/4627

(57) ABSTRACT

A system and method for improving installation of a prosthesis. Devices include prosthesis installation tools, prosthesis assembly tools, site preparation systems, and improved power tools used in implant site preparation.

### CROSS REFERENCE TO RELATED APPLICATIONS

This application is a continuation-in-part of U.S. patent application Ser. No. 15/202,434 which in turn claims benefit of U.S. patent application Ser. No. 62/277,294, all of which are hereby expressly incorporated by reference in their entireties for all purposes.

### FIELD OF THE INVENTION

The present invention relates generally to installation of a prosthesis, and more specifically, but not exclusively, to improvements in prosthesis placement and positioning.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Earlier patents issued to the present applicant have described problems associated with prosthesis installation, for example acetabular cup placement in total hip replacement surgery. See U.S. Pat. Nos. 9,168,154 and 9,220,612, which are hereby expressly incorporated by reference thereto in their entireties for all purposes. Even though hip replacement surgery has been one of the most successful operations, it continues to be plagued with a problem of inconsistent acetabular cup placement. Cup mal-positioning is the single greatest cause of hip instability, a major factor in polyethylene wear, osteolysis, impingement, component loosening and the need for hip revision surgery.

These incorporated patents explain that the process of cup implantation with a mallet is highly unreliable and a significant cause of this inconsistency. The patents note two specific problems associated with the use of the mallet. First is the fact that the surgeon is unable to consistently hit on the center point of the impaction plate, which causes undesirable torques and moment arms, leading to mal-alignment of the cup. Second, is the fact that the amount of force utilized in this process is non-standardized.

In these patents there is presented a new apparatus and method of cup insertion which uses an oscillatory motion to insert the prosthesis. Prototypes have been developed and continue to be refined, and illustrate that vibratory force may allow insertion of the prosthesis with less force, as well, in some embodiments, of allowing simultaneous positioning and alignment of the implant.

There are other ways of breaking down of the large undesirable, torque-producing forces associated with the discrete blows of the mallet into a series of smaller, axially aligned controlled taps, which may achieve the same result incrementally, and in a stepwise fashion to those set forth in the incorporated patents, (with regard to, for example, cup insertion without unintended divergence).

There are two problems that may be considered independently, though some solutions may address both in a single solution. These problems include i) undesirable and unpredictable torques and moment arms that are related to the primitive method currently used by surgeons, which involves manually banging the mallet on an impaction plate mated to the prosthesis and ii) non-standardized and essentially uncontrolled and unquantized amounts of force utilized in these processes.

What is needed is a system and method for improving installation of a prosthesis.

### BRIEF SUMMARY OF THE INVENTION

Disclosed is a system and method for improving installation of a prosthesis. The following summary of the invention is provided to facilitate an understanding of some of the technical features related to prosthesis assembly and installation, and is not intended to be a full description of the present invention. A full appreciation of the various aspects of the invention can be gained by taking the entire specification, claims, drawings, and abstract as a whole. The present invention is applicable to other prosthesis in addition to acetabular cups, other modular prosthesis in addition to assembly of modular femoral and humeral prosthesis, and to other alignment and navigation systems in addition to referenced light guides.

An embodiment of the present invention may include axial alignment of force transference, such as, for example, an axially sliding hammer moving between stops to impart a non-torqueing installation force. There are various ways of motivating and controlling the sliding hammer, including a magnitude of transferred force. Optional enhancements may include pressure and/or sound sensors for gauging when a desired depth of implantation has occurred.

Other embodiments include adaptation of various devices for accurate assembly of modular prostheses, such as those that include a head accurately impacted onto a trunion taper that is part of a stem or other element of the prosthesis.

Still other embodiments include an alignment system to improve site preparation, such as, for example, including a projected visual reference of a desired orientation of a tool and then having that reference marked and available for use during operation of the tool to ensure that the alignment remains proper throughout its use, such as during a reaming operation.

Further embodiments include enhancement of various tools, such as those used for cutting, trimming, drilling, and the like, with ultrasonic enhancement to make the device a better cutting, trimming, drilling, etc. device to enable its use with less strength and with improved accuracy.

Any of the embodiments described herein may be used alone or together with one another in any combination. Inventions encompassed within this specification may also include embodiments that are only partially mentioned or alluded to or are not mentioned or alluded to at all in this brief summary or in the abstract. Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies or just one deficiency that may be discussed in the specification, and some embodiments may not address any of these deficiencies.

Other features, benefits, and advantages of the present invention will be apparent upon a review of the present disclosure, including the specification, drawings, and claims.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1-FIG. 6 illustrate embodiments including installation of a prosthesis, including installation into living bone;

FIG. 1 illustrates an embodiment of the present invention for a sliding impact device;

FIG. 2 illustrates a lengthwise cross-section of the embodiment illustrated in FIG. 1 including an attachment of a navigation device;

FIG. 3 illustrates a cockup mechanical gun embodiment, an alternative embodiment to the sliding impact device illustrated in FIG. 1 and FIG. 2;

FIG. 4 illustrates an alternative embodiment to the devices of FIG. 1-3 including a robotic structure;

FIG. 5 illustrates an alternative embodiment to the devices of FIG. 1-4 including a pressure sensor to provide feedback;

FIG. 6 illustrates an alternative embodiment to the feedback system of FIG. 5 including a sound sensor to provide feedback for the embodiments of FIG. 1-5;

FIG. 7-FIG. 10 illustrate prosthesis assembly embodiments including use of variations of the prosthesis installation embodiments of FIG. 1-FIG. 6, such as may be used to reduce a risk of trunionosis;

FIG. 7 illustrates a modular prosthesis and assembly tools;

FIG. 8 illustrates a femoral head to be assembled onto a trunion attached to a femoral stem;

FIG. 9 illustrates alignment of an installation device with the femoral head for properly aligned impaction onto the trunion, such as an embodiment of FIG. 1-FIG. 6 adapted for this application;

FIG. 10 illustrates use of a modified vibratory system for assembly of the modular prosthesis;

FIG. 11-FIG. 12 illustrate an improvement to site preparation for an installation of a prosthesis;

FIG. 11 illustrates an environment in which a prosthesis is installed highlighting problem with site preparation; and

FIG. 12 illustrates an alignment system for preparation and installation of a prosthesis;

FIG. 13 illustrates modified surgical devices incorporating vibratory energy as at least an aid to mechanical preparation;

FIG. 14-FIG. 16 relate to a first particular implementation of a mechanical BMD for controlled axial impact;

FIG. 14 illustrates a perspective view of the particular BMD;

FIG. 15 illustrates a first actuator for use with the particular BMD of FIG. 14; and

FIG. 16 illustrates a second actuator for use with the particular BMD of FIG. 14; and

FIG. 17 illustrates a cross-sectional view of an impact energy control mechanism (spring preload) as may be used in the particular BMD of FIG. 14;

FIG. 18 illustrates an internal view of an impact energy control mechanism (spring preload) as may be used in the particular BMD of FIG. 14;

FIG. 19 illustrates cross-sectional view of an impact energy control mechanism (friction) as may be used in the particular BMD of FIG. 14;

FIG. 20 illustrates an internal view of an impact energy control mechanism (friction) as may be used in the particular BMD of FIG. 14;

FIG. 21 illustrates a close-up detail of an impact energy control mechanism (friction), ball-detent as may be used in the particular BMD of FIG. 14;

FIG. 22 illustrates a bottom view of an impact energy control mechanism (friction) as may be used in the particular BMD of FIG. 14;

FIG. 23-FIG. 24 relate to a second particular implementation of a mechanical BMD for controlled axial impact;

FIG. 23 illustrates a hand-operated slide hammer implementation for the mechanical BMD; and

FIG. 24 illustrates an exploded view of the mechanical BMD of FIG. 23;

FIG. 25-FIG. 27 relate to a third particular implementation of a mechanical BMD for controlled axial impact;

FIG. 25 illustrates a pneumatically-operated slide hammer implementation for the mechanical BMD;

FIG. 26 illustrates an internal view of the mechanical BMD of FIG. 25;

FIG. 27 illustrates an exploded view of the mechanical BMD of FIG. 25; and

FIG. 28 illustrates a detail view of the pneumatic engine for the BMD of FIG. 25.

### DETAILED DESCRIPTION OF THE INVENTION

Embodiments of the present invention provide a system and method for improving installation of a prosthesis. The following description is presented to enable one of ordinary skill in the art to make and use the invention and is provided in the context of a patent application and its requirements.

Various modifications to the preferred embodiment and the generic principles and features described herein will be readily apparent to those skilled in the art. Thus, the present invention is not intended to be limited to the embodiment shown but is to be accorded the widest scope consistent with the principles and features described herein.

Definitions

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The following definitions apply to some of the aspects described with respect to some embodiments of the invention. These definitions may likewise be expanded upon herein.

As used herein, the term “or” includes “and/or” and the term “and/or” includes any and all combinations of one or more of the associated listed items. Expressions such as “at least one of,” when preceding a list of elements, modify the entire list of elements and do not modify the individual elements of the list.

As used herein, the singular terms “a,” “an,” and “the” include plural referents unless the context clearly dictates otherwise. Thus, for example, reference to an object can include multiple objects unless the context clearly dictates otherwise.

Also, as used in the description herein and throughout the claims that follow, the meaning of “in” includes “in” and “on” unless the context clearly dictates otherwise. It will be understood that when an element is referred to as being “on” another element, it can be directly on the other element or intervening elements may be present therebetween. In contrast, when an element is referred to as being “directly on” another element, there are no intervening elements present.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common properties.

As used herein, the term “adjacent” refers to being near or adjoining. Adjacent objects can be spaced apart from one another or can be in actual or direct contact with one another. In some instances, adjacent objects can be coupled to one another or can be formed integrally with one another.

As used herein, the terms “connect,” “connected,” and “connecting” refer to a direct attachment or link. Connected objects have no or no substantial intermediary object or set of objects, as the context indicates.

As used herein, the terms “couple,” “coupled,” and “coupling” refer to an operational connection or linking. Coupled objects can be directly connected to one another or can be indirectly connected to one another, such as via an intermediary set of objects.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%.

As used herein, the terms “substantially” and “substantial” refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

As used herein, the terms “optional” and “optionally” mean that the subsequently described event or circumstance may or may not occur and that the description includes instances where the event or circumstance occurs and instances in which it does not.

As used herein, the term “size” refers to a characteristic dimension of an object. Thus, for example, a size of an object that is spherical can refer to a diameter of the object. In the case of an object that is non-spherical, a size of the non-spherical object can refer to a diameter of a corresponding spherical object, where the corresponding spherical object exhibits or has a particular set of derivable or measurable properties that are substantially the same as those of the non-spherical object. Thus, for example, a size of a non-spherical object can refer to a diameter of a corresponding spherical object that exhibits light scattering or other properties that are substantially the same as those of the non-spherical object. Alternatively, or in conjunction, a size of a non-spherical object can refer to an average of various orthogonal dimensions of the object. Thus, for example, a size of an object that is a spheroidal can refer to an average of a major axis and a minor axis of the object. When referring to a set of objects as having a particular size, it is contemplated that the objects can have a distribution of sizes around the particular size. Thus, as used herein, a size of a set of objects can refer to a typical size of a distribution of sizes, such as an average size, a median size, or a peak size.

Embodiments of the present invention may include one of more solutions to the above problems. The incorporated U.S. Pat. No. 9,168,154 includes a description of several embodiments, sometimes referred to herein as a BMD3 device, some of which illustrate a principle for breaking down large forces associated with the discrete blows of a mallet into a series of small taps, which in turn perform similarly in a stepwise fashion while being more efficient and safer. The BMD3 device produces the same displacement of the implant without the need for the large forces from the repeated impacts from the mallet. The BMD3 device may allow modulation of force required for cup insertion based on bone density, cup geometry, and surface roughness. Further, a use of the BMD3 device may result in the acetabulum experiencing less stress and deformation and the implant may experience a significantly smoother sinking pattern into the acetabulum during installation. Some embodiments of the BMD3 device may provide a superior approach to these problems, however, described herein are two problems that can be approached separately and with more basic methods as an alternative to, or in addition to, a BMD3 device. An issue of undesirable torques and moment arms is primarily related to the primitive method currently used by surgeons, which involves manually banging the mallet on the impaction plate. The amount of force utilized in this process is also non-standardized and somewhat out of control.

With respect to the impaction plate and undesirable torques, an embodiment of the present invention may include a simple mechanical solution as an alternative to some BMD3 devices, which can be utilized by the surgeon's hand or by a robotic machine. A direction of the impact may be directed or focused by any number of standard techniques (e.g., A-frame, C-arm or navigation system). Elsewhere described herein is a refinement of this process by considering directionality in the reaming process, in contrast to only considering it just prior to impaction. First, we propose to eliminate the undesirable torques by delivering the impacts by a sledgehammer device or a structure (e.g., hollow cylindrical mass) that travels over a stainless rod.

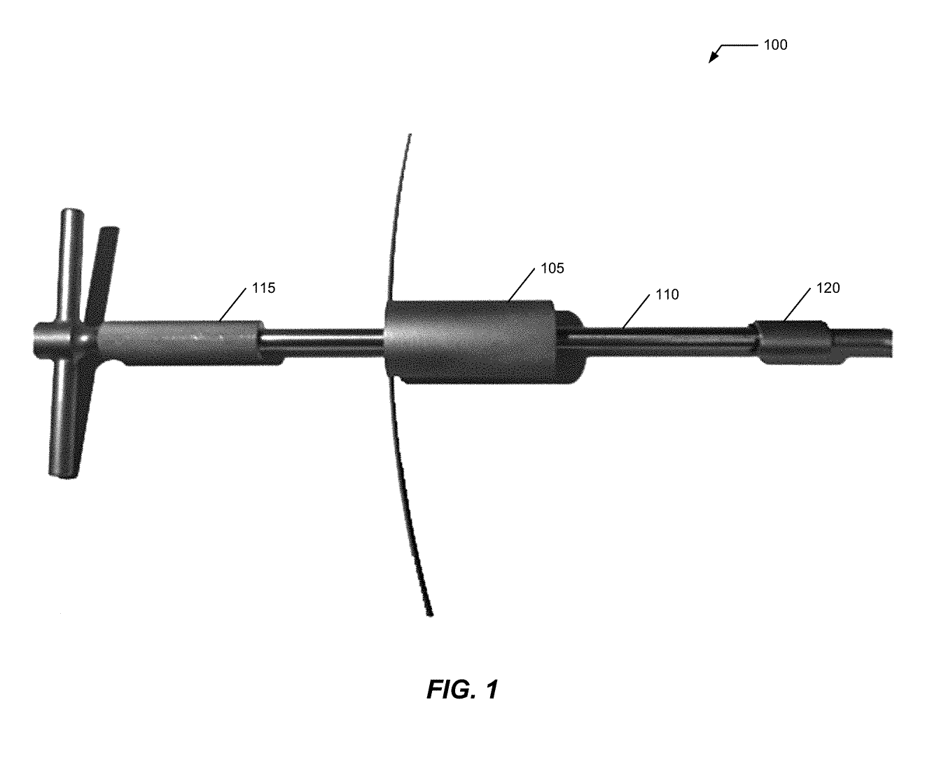

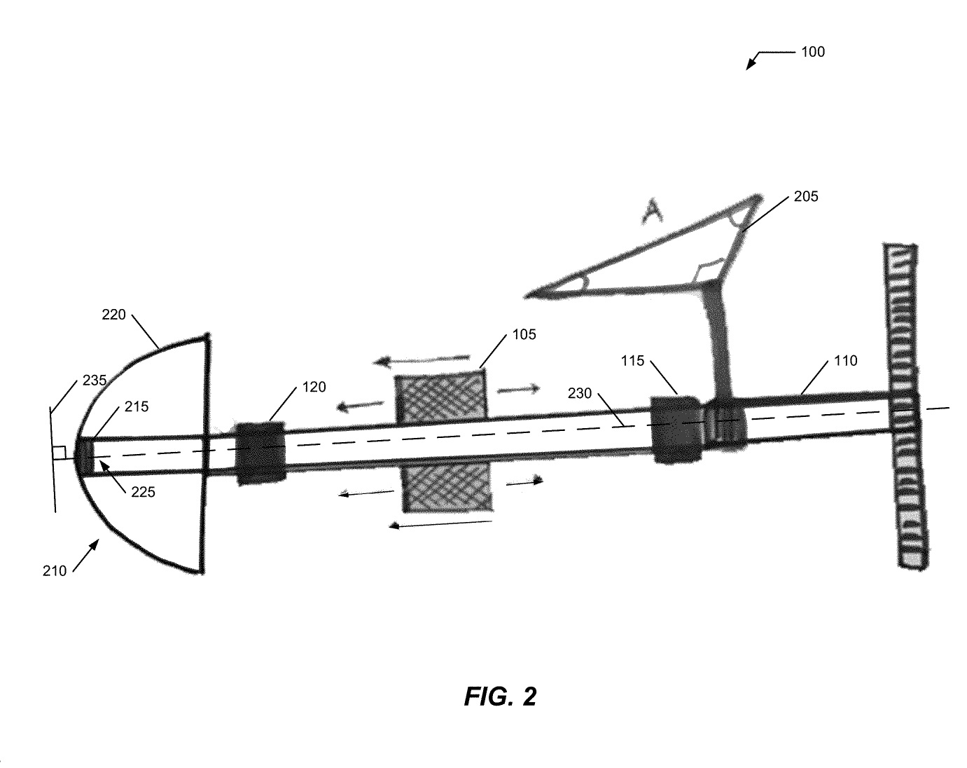

FIG. 1 illustrates an embodiment of the present invention for a sliding impact device 100, and FIG. 2 illustrates a lengthwise cross-section of sliding impact device 100 including an attachment of a navigation device 205.

Device 100 includes a moveable hammer 105 sliding axially and freely along a rod 110. Rod 110 includes a proximal stop 115 and distal stop 120. These stops that may be integrated into rod 110 to allow transference of force to rod 110 when hammer 105 strikes distal stop 120. At a distal end 210 of rod 110, device 100 includes an attachment system 215 for a prosthesis 220. For example, when prosthesis 220 includes an acetabular cup having a threaded cavity 225, attachment system 215 may include a complementary threaded structure that screws into threaded cavity 225. The illustrated design of device 100 allows only a perfect axial force to be imparted. The surgeon cannot deliver a blow to the edge of an impaction plate. Therefore the design of this instrument is in and of itself protective, eliminating a problem of “surgeon's mallet hitting on the edge of the impaction plate” or other mis-aligned force transference, and creating undesirable torques, and hence unintentional mal-alignment of prosthesis 220 from an intended position/orientation.

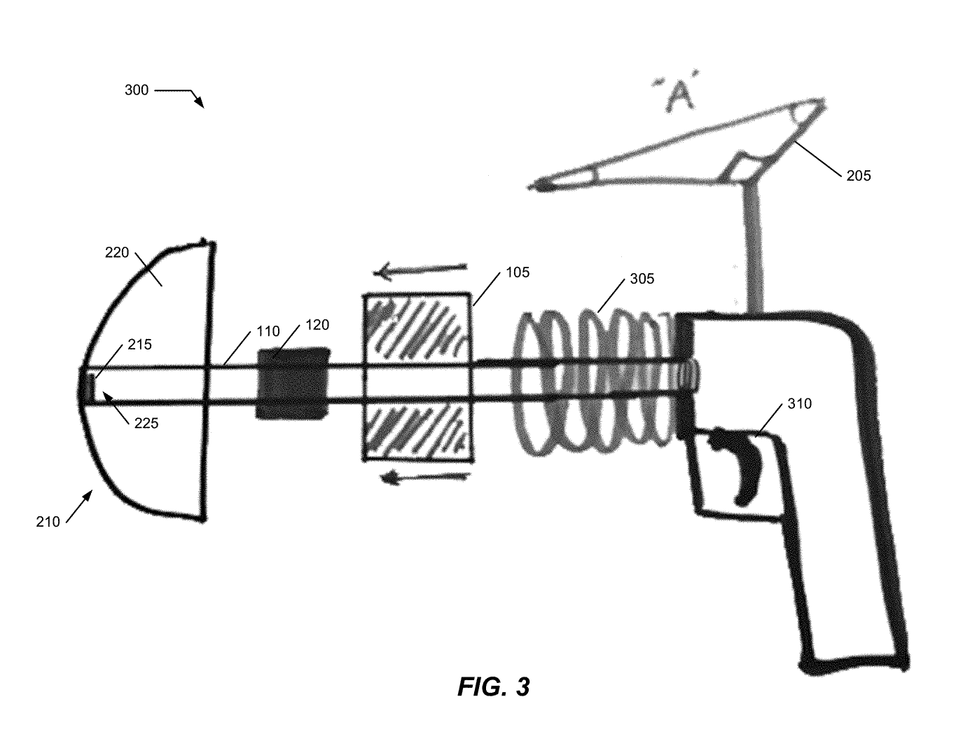

A longitudinal axis 230 extends through the ends of rod 110. Attachment system 215 aligns prosthesis 220 to axis 230 when rod 110 is coupled to threaded cavity 225. An apex of prosthesis 220 (when it generally defines a hollow semispherical shell) supports a structure that defines threaded cavity 225 and that structure may define a plane 235 that may be tangent to the apex, with plane 235 about perpendicular to axis 230 when rod 110 engages prosthesis 220. Operation of device 100 is designed to deliver only axial (e.g., aligned with axis 230 and thus non-torqueing) forces to prosthesis 220. Other embodiments illustrated in FIG. 3-FIG. 6 may be similarly configured.

FIG. 3 illustrates a cockup mechanical gun 300 embodiment, an alternative embodiment to the sliding impact device illustrated in FIG. 1 and FIG. 2. An alternate embodiment includes cockup mechanical gun 300 that uses the potential energy of a cocked up spring 305 to create an axially aligned impaction force. Hammer 105 is drawn back and spring 305 is locked until an operator actuates a trigger 310 to release spring 305 and drive hammer 105 along rod 110 to strike distal stop 120 and transfer an axially aligned impacting force to prosthesis 220.

Each pull of trigger 310 creates the same predetermined fixed unit of force (some alternatives may provide a variably predetermined force). The surgeon cannot deliver a misaligning impact to an impaction plate with this design.

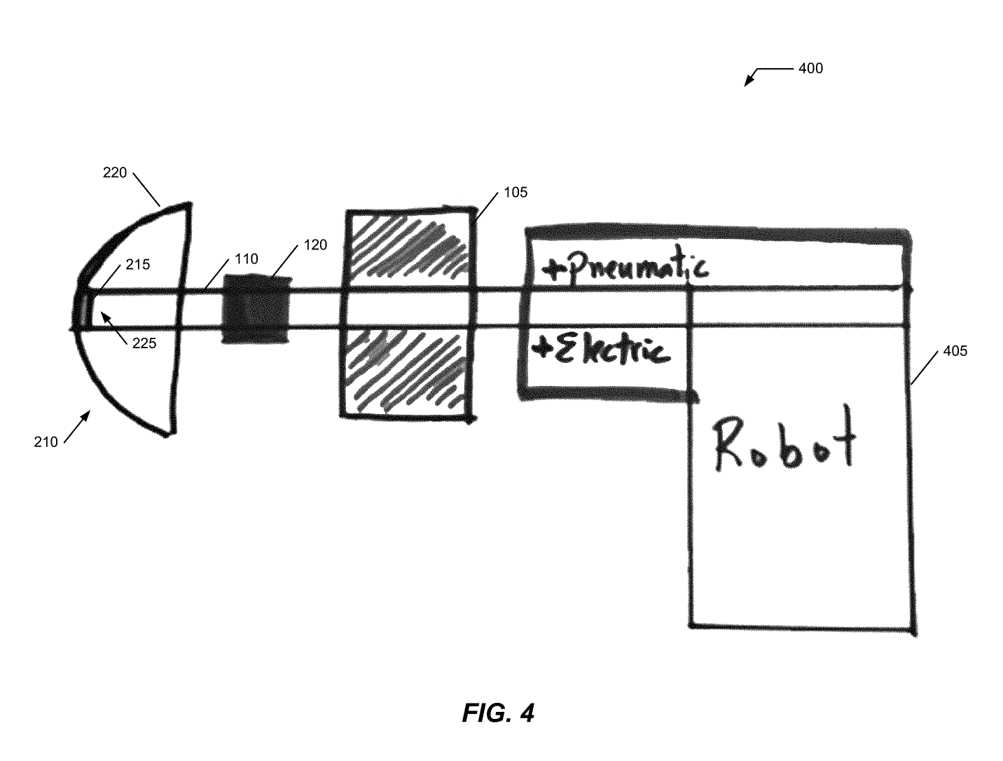

FIG. 4 illustrates an alternative robotic device 400 embodiment to the devices of FIG. 1-3 including a robotic control structure 405. For example, device 100 and/or device 300 may be mounted with robot control structure 405 and the co-axial impacts may be delivered mechanically by a robotic tool using pneumatic or electric energy.

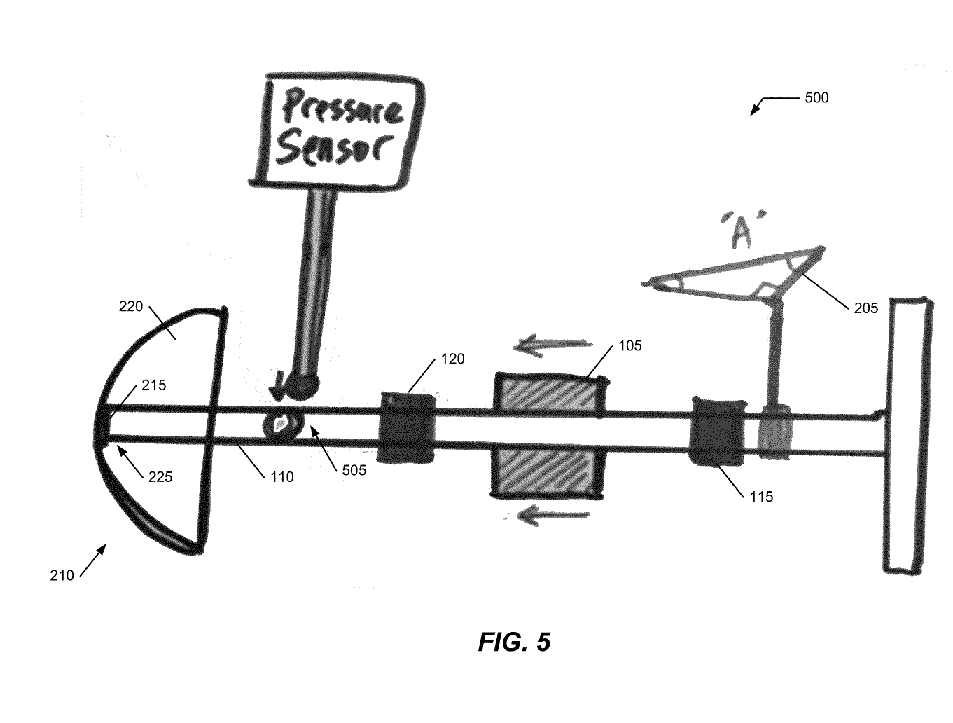

FIG. 5 illustrates an alternative embodiment 500 to the devices of FIG. 1-4 including a pressure sensor 505 to provide feedback during installation. With respect to management of the force required for some of these tasks, it is noted that with current techniques (the use of the mallet) the surgeon has no indication of how much force is being imparted onto the implant and/or the implant site (e.g., the pelvis). Laboratory tests may be done to estimate what range of force should be utilized in certain age groups (as a rough guide) and then fashioning a device 500, for example a modified sledgehammer 100 or cockup gun 300 to produce just the right amount of force. Typically the surgeon may use up to 2000 N to 3000 N of force to impact a cup into the acetabular cavity. Also, since some embodiments cannot deliver the force in an incremental fashion as described in association with the BMD3 device, device 500 includes a stopgap mechanism. Some embodiments of the BMD3 device have already described the application of a sensor in the body of the impaction rod. Device 500 includes sensing system/assembly 505 embedded in device 500, for example proximate rod 110 near distal end 210, and used to provide valuable feedback information to the surgeon. Pressure sensor 505 can let the surgeon know when the pressures seems to have maximized, whether used for the insertion of an acetabular cup, or any other implant including knee and shoulder implants and rods used to fix tibia and femur fractures. When pressure sensor 505 is not showing an advance or increase in pressure readings and has plateaued, the surgeon may determine it is time to stop operation/impacting. An indicator, for example an alarm can go off or a red signal can show when maximal peak forces are repeatedly achieved. As noted above, the incorporated patents describe a presence of a pressure sensor in an installation device, the presence of which was designed as part of a system to characterize an installation pulse pattern communicated by a pulse transfer assembly. The disclosure here relates to a pressure sensor provided not to characterize the installation pulse pattern but to provide an in situ feedback mechanism to the surgeon as to a status of the installation, such as to reduce a risk of fracturing the installation site. Some embodiments may also employ this pressure sensor for multiple purposes including characterization of an applied pulse pattern such as, for example, when the device includes automated control of an impacting engine coupled to the hammer. Other embodiments of this invention may dispose the sensor or sensor reading system within a handle or housing of the device rather than in the central rod or shaft.

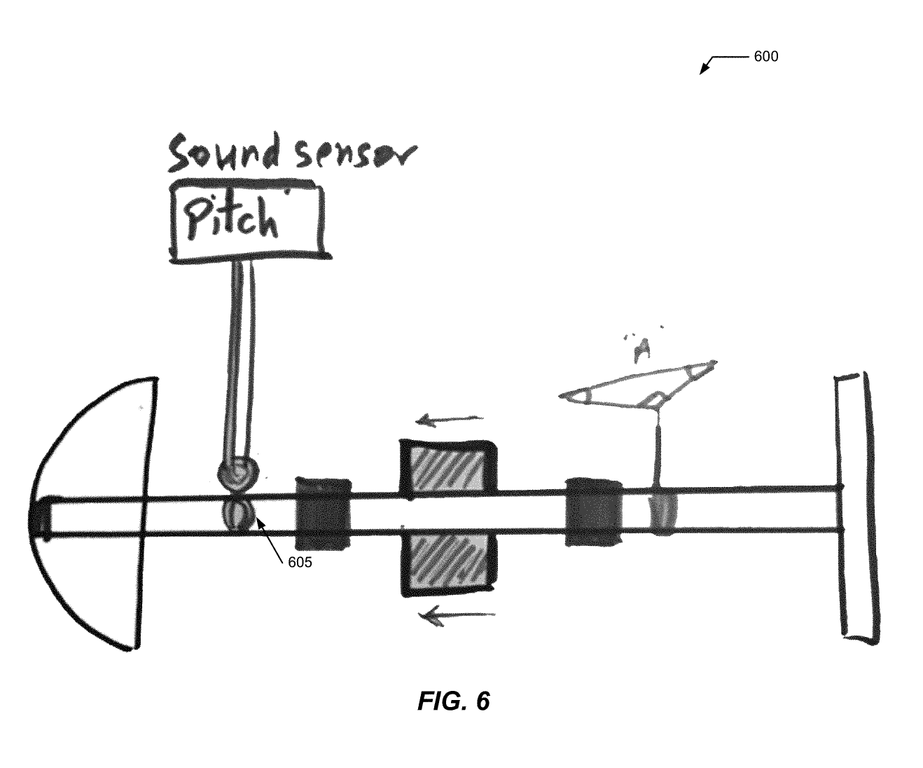

FIG. 6 illustrates an alternative device 600 embodiment to the feedback system of FIG. 5 including a sound sensor 605 to provide feedback for the embodiments of FIG. 1-5. Surgeons frequently use a change in pitch (sound) to gauge whether an implant (e.g., the cup) has “bottomed out” (an evaluation of a “seatedness” of the implant) and device 600 includes sound sensor 605 either attached or coupled to rod 110 or otherwise disposed separately in the operating room. Sound sensor system/assembly 605 may be used in lieu of, or in addition to, pressure sensor system/assembly 505 illustrated in FIG. 5.

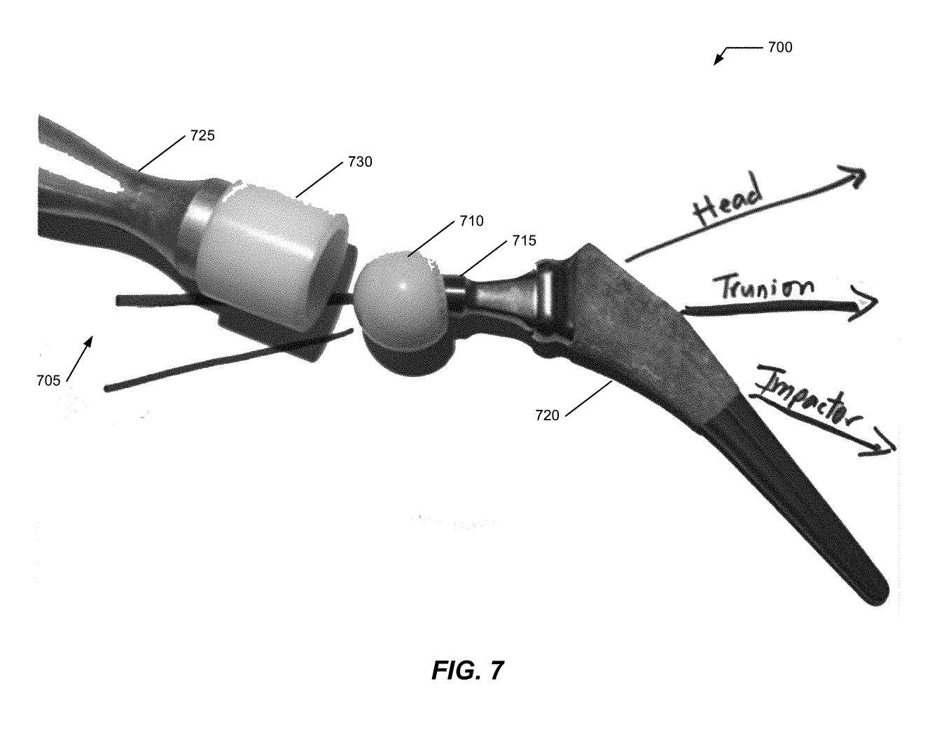

FIG. 7-FIG. 10 illustrate prosthesis assembly embodiments including use of variations of the prosthesis installation embodiments of FIG. 1-FIG. 6, such as may be used to reduce a risk of trunionosis or for other advantage. FIG. 7 illustrates a modular prosthesis 700 and assembly tool 705. Prosthesis 700 includes a head 710 and a trunion taper 715 at an end of a stem 720 (e.g., a femoral stem for supporting a ball head to fit within an acetabular cup used in a total hip replacement procedure). During the procedure, the surgeon assembles prosthesis 700 by using tool 705 which may include an impact rod 725 attached to a head coupler 730. The surgeon uses tool 705 to drive head 710 onto trunion taper 715 which conventionally includes a free mallet striking tool 705. Such a procedure may be prone to the similar problems as installation of a prosthesis into an implant site, namely application of off-axis torqueing forces and an uncertainty of applied force and completion of assembly.

It is believed that even a 0.1 degree mal-alignment on head 710 on trunion taper 715 may lead to progressive wear and metalosis. Variations of the embodiments of devices illustrated in FIG. 1-FIG. 6 and its associated content may be developed to help resolve this problem. In the case of “non-torqueing axiallity” of forces from an assembly device, a bore of the head may define an axis, the trunion taper may define an axis, with the assembly device aligning these axes and then applying its forces in co-axial alignment with these co-axially aligned axes. Such an embodiment may reduce or eliminate any force-responsive rotations of the head with respect to the taper as the head is seated into position by the assembly device.

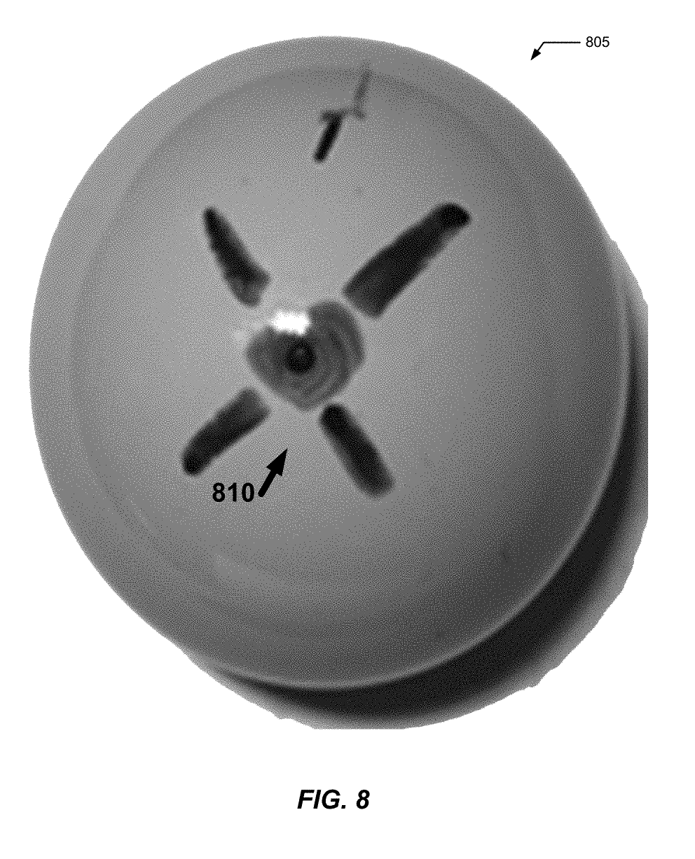

FIG. 8 illustrates a femoral head 805, a variation of head 710 illustrated in FIG. 7, to be assembled onto trunion taper 715 that is coupled to femoral stem 720. A center dot 810 may be placed on femoral (or humeral) head 805 to be impacted using tool 705.

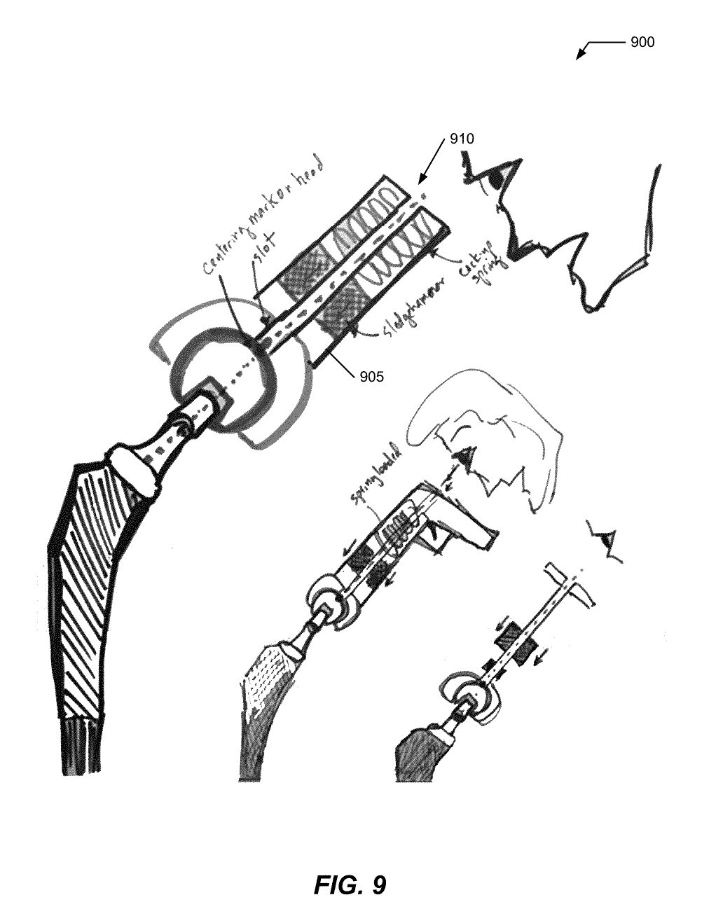

FIG. 9 illustrates alignment of an installation device 900, a variation of any of devices 100-600, with femoral head 805 for properly aligned impaction onto trunion taper 715, such as an embodiment of FIG. 1-FIG. 6 adapted for this application. Such adaptation may include, for example, an axial channel 910 to view dot 810, and align force transference, prior to operation of hammer 105.

Dot 810 can be aligned with an impactor/device/gun. Once axial alignment, such as through the sight channel, has been confirmed, a sledgehammer, a cockup gun, or other similar device can bang the impactor onto femoral (humeral) head 805 to impact it on trunion taper 715. The co-axiality of the head and the device can be confirmed visually (for example, through a hollow cylinder that comprises a center shaft of the device) or with a variety of electronic and laser methods.

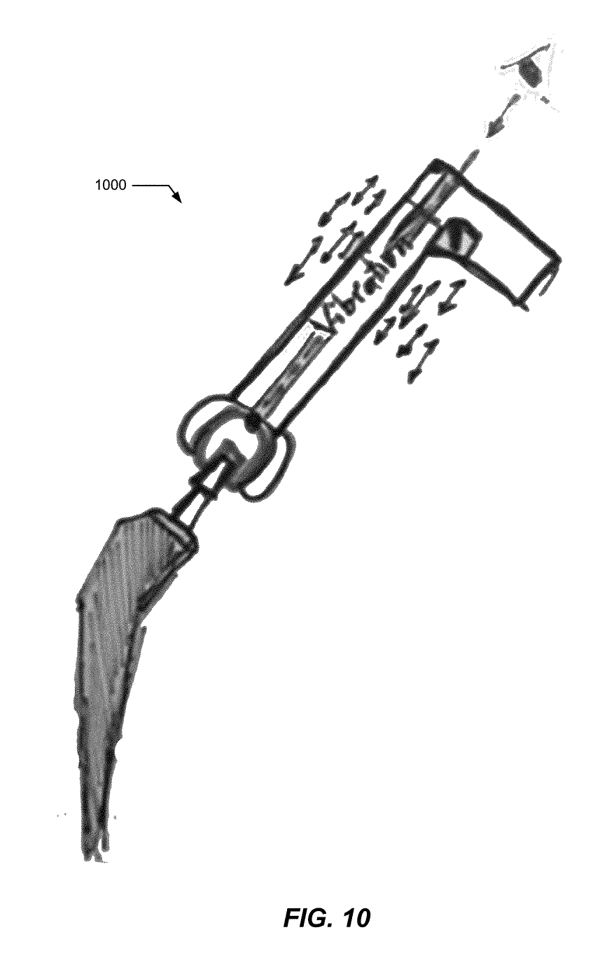

FIG. 10 illustrates use of a modified vibratory system 1000, a variation of installation device 900 for assembly of the modular prosthesis illustrated in FIG. 7. Alternatively to device 900, a variation of the BMD3 device can be used to insert the femoral and humeral heads 710 onto trunion taper 715. For example, a version of the BMD3 device where femoral head 710 is grasped by a “vibrating gun” and introduced methodically and incrementally onto trunion taper 715. Since there are no large forces being applied to the head/trunion junction, there is essentially no possibility, or a reduced possibility, of head 710 seating onto trunion taper 715 in a misaligned fashion. It would be possible to use the same technique of marking the center of head 710 and lining it up with trunion taper 715 and device axially before operating the device.

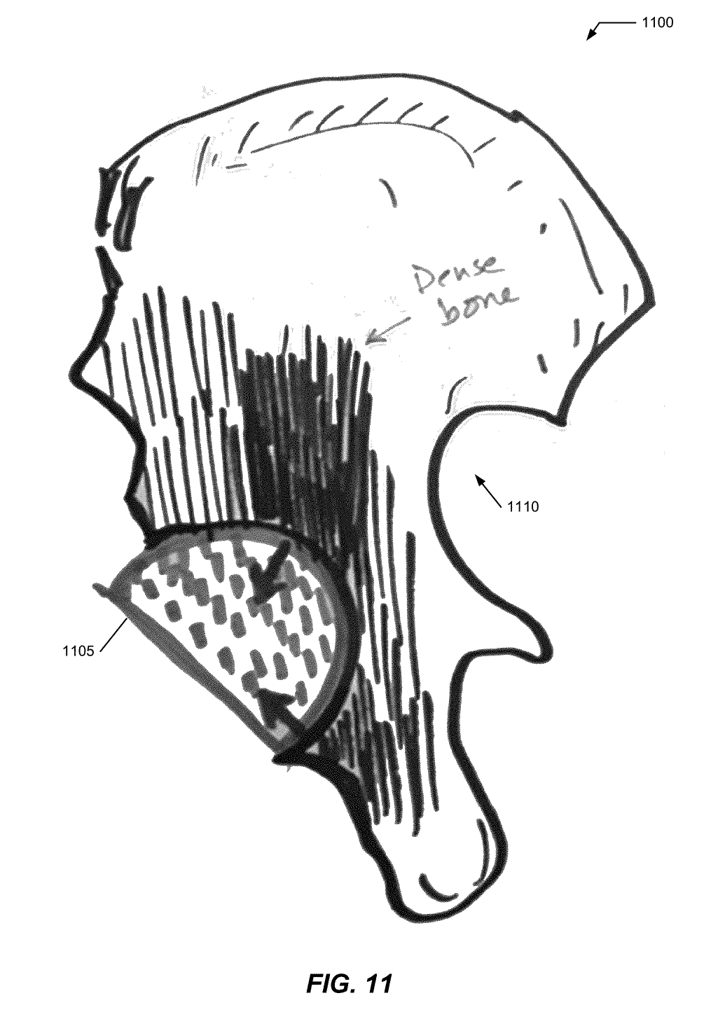

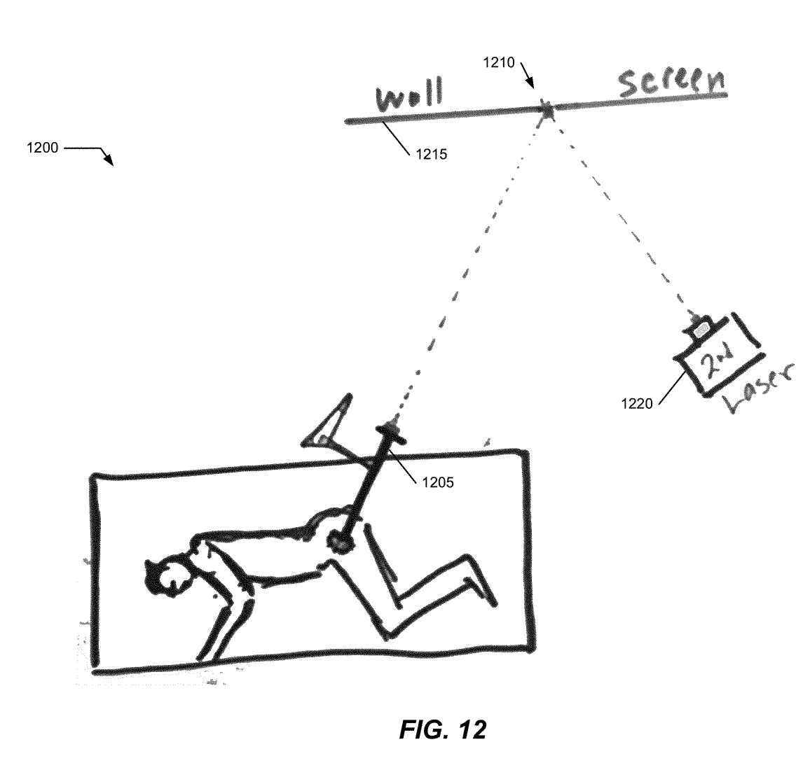

FIG. 11-FIG. 12 illustrate an improvement to site 1100 preparation for an installation of a prosthesis 1105. FIG. 11 illustrates an environment 1100 in which prosthesis 1105 is installed highlighting a problem with site preparation for a prosthesis installation procedure having variable density bone (line thickness/separation distance reflecting variable bone density) of acetabulum 1110.

There is a secondary problem with the process of acetabular preparation and implantation that leads to cup mal-alignment. Currently, during the process of acetabular reaming, surgeons make several assumptions. One common assumption is that the reamer is fully seated in a cavity and surrounded on all sides by bone. Another common assumption is that the bone that is being reamed is uniform in density. Imagine a carpenter that is preparing to cut a piece of wood with a saw. Now imagine that parts of this piece of wood are embedded with cement and some parts of the piece of wood are hollow and filled with air. The carpenter's saw will not produce a precise cut on this object. Some parts are easy to cut and some parts are harder to cut. The saw blades skives and bends in undesirable ways. A similar phenomenon happens in acetabular preparation with a reamer and when performing the cuts for knee replacement with a saw. With respect to the acetabulum, the side of the cavity that is incomplete (side of the reamer that is uncovered) will offer less resistance to the reamer and therefor the reamer preferentially reams towards the direction of the uncovering. Second, the reamer cuts the soft bone much more easily than the dense and sclerotic bone, so the reamer moves away from the sclerotic bone and moves toward the soft bone. From a machining perspective, the reaming and preparation of the acetabulum may not be concentric or precise. This maybe a significant factor in the surgeon's inability to impact the cup in the desired location

FIG. 12 illustrates an alignment system 1200 for preparation and installation of a prosthesis to help address/minimize this effect. A first step that can be taken is to include directionality into the process of reaming at the outset, and not just at the last step during impaction. Current technique allows the surgeon to ream the cup haphazardly moving the reamer handle in all directions, being ignorantly unaware that he is actually creating a preference for the sinking path of the acetabular implant. Ultimately the direction in which the surgeon reams may in fact be determining the position/path of the final implant. The surgeon then impacts the cup using the traditional A-frame or any of the currently used intra-operative measurement techniques such as navigation or fluoroscopy. These methods provide information about the position of the cup either as it is being implanted or after the implantation has occurred. None of these techniques predetermine the cup's path or function to guide the cup in the correct path.

Proposed is a method and a technique to eliminate/reduce this problem. Before the surgeon begins to ream the acetabulum, the reamer handle should be held, with an A-frame attached, in such a way to contemplate the final position of the reamer and hence the implant, (e.g., hold the reamer in 40 degree abduction and 20 degree anteversion reaming is started). This step could also be accomplished with navigation or fluoroscopy. The surgeon could, for example, immediately mark this position on a screen or the wall in the operating room as described below and as illustrated in FIG. 12. After the anticipated position of the reamer is marked, the surgeon can do whatever aspect of reaming that needs to be done. For example the first reaming usually requires medialization in which the reamer is directed quite vertically to ream in to the pulvinar. Typically three or four reamings are done. First, the acetabular cavity is medialized. The other reamings function to get to the subchondral bone in the periphery of the acetabulum. One solution may be that after each reaming, the reamer handle be held in the final anticipated position of the implant. In some cases it may be difficult to have an A-frame attached to every reamer and to estimate the same position of the reamer in the operating space accurately with the A-frame.

An alternative to that is also proposed to address this process. For example, at a proximal end of the reamer shaft handle will be placed a first reference system 1205, for example a laser pointer. This laser pointer 1205 will project a spot 1210 either on a wall or on a screen 1215, a known distance from the operating room table. That spot 1210 on wall 1215 (or on the screen) is then marked with another reference system 1220, for example a second independent laser pointer that sits on a steady stand in the operating room. Thereafter manipulating the shaft handle so that the first reference system has the desired relationship, example co-aligned, with the second reference system, the surgeon knows that the device attached to the handle has the desired orientation. So when the first reamer is held in the anticipated and desired final alignment of the implant (e.g., 40 degree abduction, 20 degree anteversion for many preferred installation angles of an acetabular cup), the laser pointer at the proximal end of the reamer handle projects a spot on the wall or screen. That spot is marked with the second stationary laser, and held for the duration of the case. All subsequent reamings will therefore not require an A-frame to get a sense of the proper alignment and direction of the reamer. The surgeon assures that no matter how he moves the reamer handle in the process of reaming of the acetabulum, that the reaming finishes with the reamer handle (laser pointer) pointing to the spot on the wall/screen. In this manner, directionality is assured during the reaming process. In this way the sinking path of the actual implant is somewhat predetermined. And no matter what final intra-operative monitoring technique is used (A-frame, C-Arm, Navigation) that the cup will likely seat/sink more closely to the desired final position.

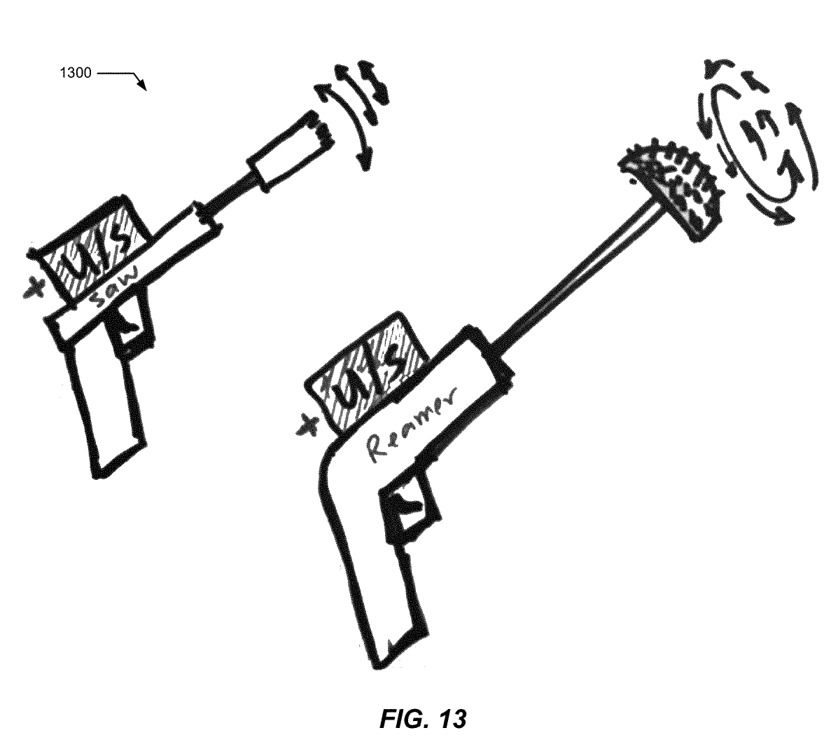

FIG. 13 illustrates modified surgical devices 1300 incorporating vibratory energy as at least an aid to mechanical preparation. Also proposed herein is another concept to address a problem associated with non-concentric reaming of the acetabulum caused by variable densities of the bone and the uncovering of the reamer. Imagine the same carpenter has to cut through a construct that is made out of wood, air, and cement. The carpenter does not know anything about the variable densities of this construct. There are two different saws available: one that cuts effectively through wood only, and ineffectively through the cement. Also available is a second saw that cuts just as effectively through cement as wood. Which of these saws would improve a chance of producing a more precise cut? Proposed is a mixing of ultrasonic energy with the standard oscillating saw and the standard reamer. In effect any oscillating equipment used in orthopedics, including the saw, reamer, drill, and the like may be made more precise in its ability to cut and prepare bone with the addition of ultrasonic energy. This may feel dangerous and counterintuitive to some, however, the surgeon typically applies a moderate amount of manual pressure to the saw and reamers, without being aware, which occasionally causes tremendous skiving, bending and eccentric reaming. An instrument that does not requires the surgeon's manual force maybe significantly safer and as well as more precise and effective.

A further option includes disposition of a sensor in the shaft of the ultrasonic reamers and saws so that the surgeon can ascertain when hard versus soft bone is being cut, adding a measure of safety by providing a visual numerical feedback as to the amount of pressure being utilized. This improvement (the ability to cut hard and soft bone with equal efficacy) will have tremendous implications in orthopedic surgery. When the acetabular cavity is prepared more precisely, with significantly lower tolerances, especially when directionality is observed, the acetabular implant (cup) may more easily follow the intended sinking path.

Other applications of this concept could be very useful. Pressfit and ingrowth fixation in total knee replacements in particular (as well as ankle, shoulder and other joints to a lesser degree) are fraught with problems, particularly that of inconsistent bony ingrowth and fixation. The fact that a surgeon is unable to obtain precise cuts on the bone may be a significant factor in why the bone ingrowth technology has not gotten off the ground in joints other than the hip. The problem is typically blamed on the surgeon and his less than perfect hands. The experienced surgeon boasts that only he should be doing this operation (i.e.: non-cemented total knee replacement). This concept (a more precise saw that cuts hard and soft bone equally allowing lower tolerances) has huge potential in orthopedics, in that it can lead to elimination of the use of cement in orthopedic surgery altogether. This can spark off the growth and use of bone ingrowth technology in all aspects of joint replacement surgery which can lead to tremendous time saving in the operating room and better results for the patients.

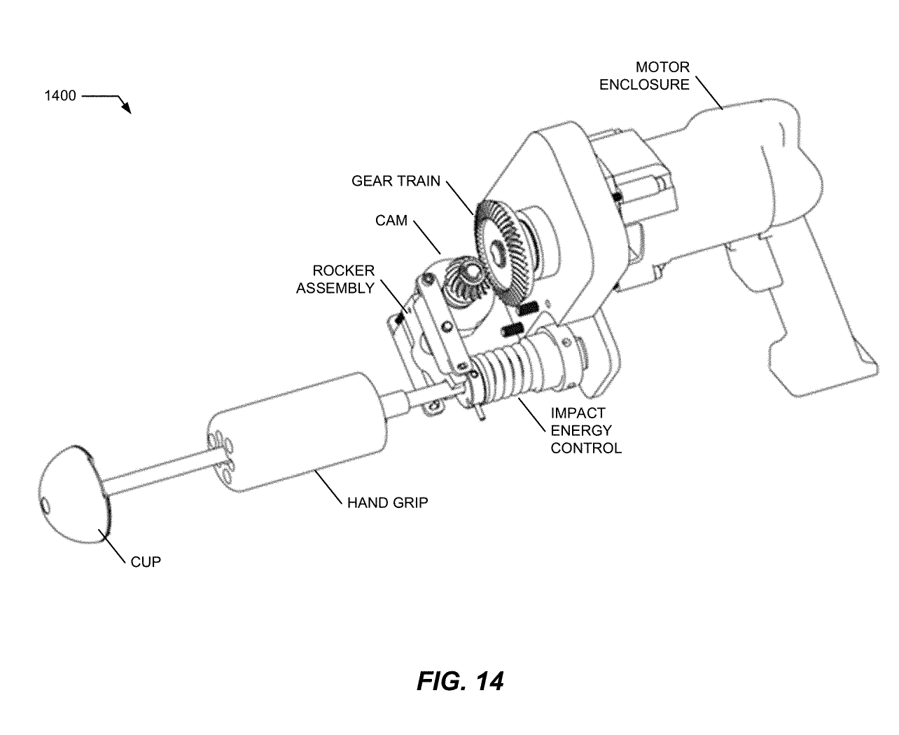

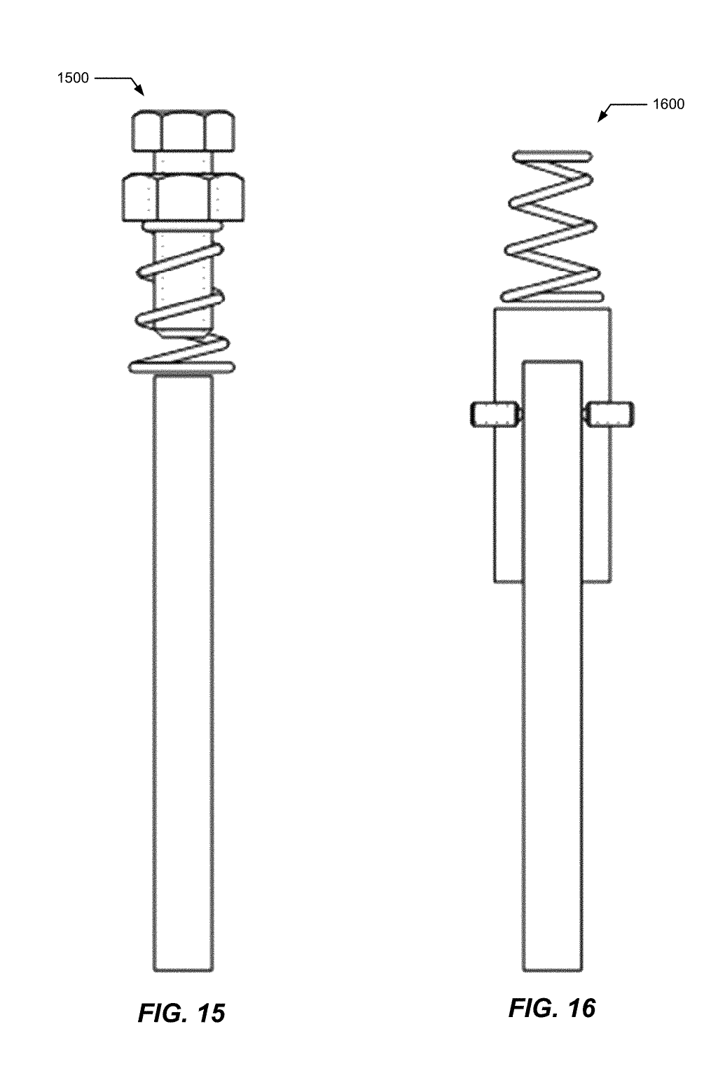

FIG. 14-FIG. 22 relate to a first particular implementation of a mechanical BMD 1400 for controlled axial impact; FIG. 14 illustrates a perspective view of BMD 1400; FIG. 15 illustrates a first impact energy control mechanism 1500 for use with the particular BMD of FIG. 14; and FIG. 16 illustrates a second impact energy control mechanism 1600 for use with the particular BMD of FIG. 14. BMD 1400 includes a motor is directly connected to a cam via a gear train. Instead of having the cam directly displace the instrument shaft, the cam an impact energy control that is positioned proximally of the shaft by means of a rocker assembly. The profile of the cam is such that the control is actuated between impacts, until a desired condition is reached and the energy is released, driving the shaft forward and generating an impact force.

The mechanism of FIG. 14 may allow a device to indirectly measure the rate of insertion of an acetabular cup while controlling the impact force being delivered to the cup as described in U.S. patent application Ser. No. 15/234,782 filed 11 Aug. 2016, the contents of which are hereby expressly incorporated by reference thereto in its entirety. The method may include a handheld instrument that would include an actuator, shaft, and cup interface. Similar to the impaction rod currently used by surgeons, the instrument would couple to an acetabular cup using an appropriate thread at the distal end of the instrument shaft. The actuator would couple to the proximal end of the instrument shaft, and create controlled impacts that would be applied to the shaft and connected cup. The magnitude of the impact would be controlled by the surgeon through a dial or other input mechanism on the device, or directly by the instrument's software.

While the cup is being inserted, each blow must reach a minimum impact force in order to overcome the static friction of the cup/bone interface. The impact force required increases as the insertion depth of the cup increases due to larger normal forces acting on the cup/bone interface (see incorporated patent application). There is a balancing act though, as larger impact forces raise the risk of fracture of surrounding bone. The goal of the surgeon is to reach a sufficient insertion depth to generate acceptable cup stability, while minimizing forces imparted to the acetabulum during the process. This area is believed to be in the beginning of the non-linear regime, as higher forces begin to have a smaller incremental benefit to cup insertion (i.e. smaller incremental insertion depth with larger forces).

There are a number of challenges with developing a tool that will aid cup insertion: 1) the insertion force plot will vary for each procedure, 2) the resulting optimal depth will vary, and 3) there is no simple way to measure the insertion depth of the cup relative to the acetabulum.

The proposed solution will instead have the actuator control the amount of energy being transmitted during each impact. This could be done in a number of ways, with two examples explained below. Both mechanisms utilize the basic arrangement of BMD 1400, but could be adapted for other implementations discussed.

Energy Impact Control Mechanism—Spring Preload:

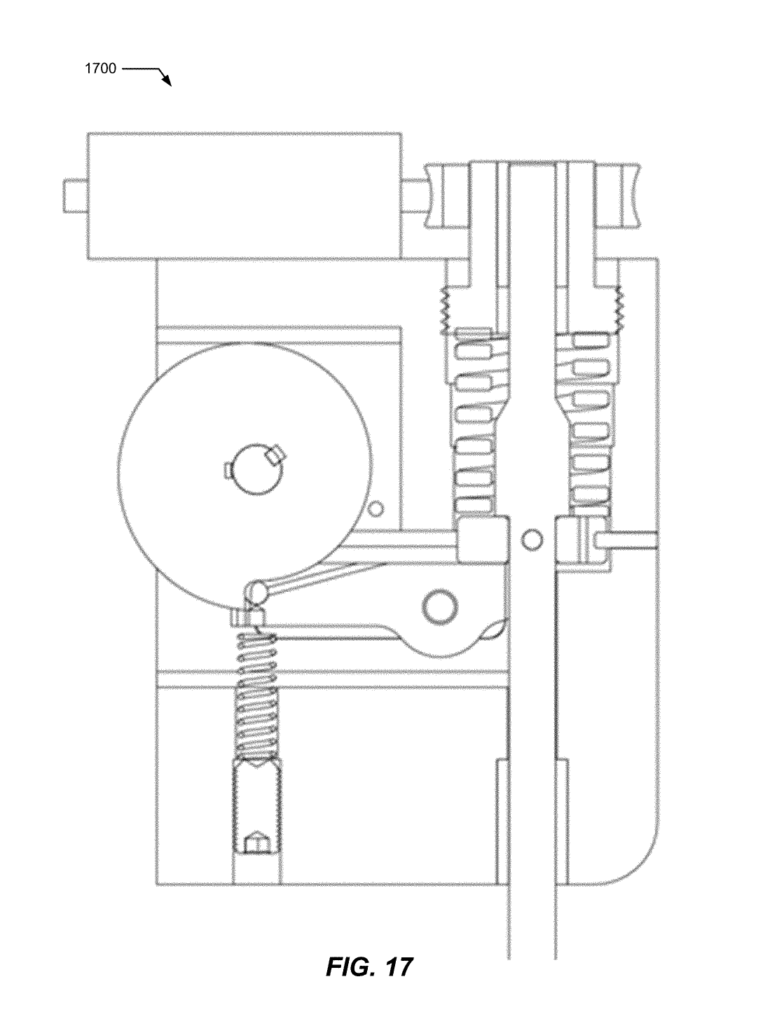

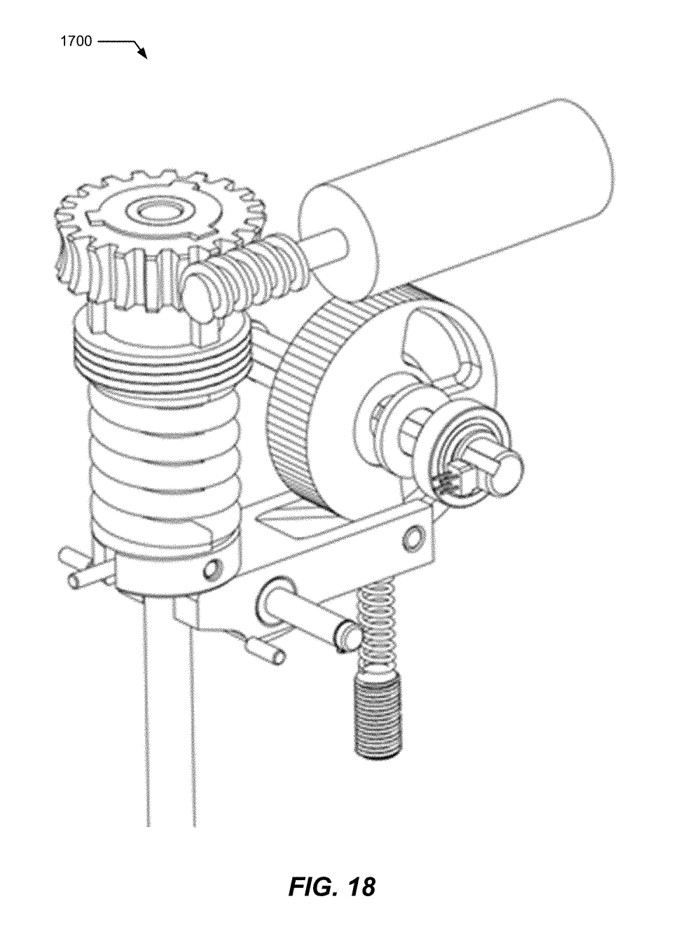

FIG. 17 illustrates a cross-sectional view of an impact energy control mechanism (spring preload) 1700 as may be used in the particular BMD of FIG. 14, and FIG. 18 illustrates an internal view of an impact energy control mechanism (spring preload) 1700 as may be used in the particular BMD of FIG. 14. The first approach would have the device compress a spring of known spring constant by retracting the instrument shaft by a fixed distance. In the figures this shaft displacement is performed via a rotating cam which in turn uses a rocker to convert the rotational motion to linear movement. The device would be able to vary the energy stored within the shaft spring for each impact by varying the amount of spring preload (i.e. the amount of spring compression immediately after an impact has occurred).

The preload is varied using a spring compression insert. The spring compression insert includes external threads which mates to the housing of the tool. A gear head is attached to the top face of the spring compression insert, which mates to a motor via a worm gear or other appropriate mechanism (e.g. chain drive, belt drive, geartrain, etc.). The vertical position of the insert relative to the shaft spring can be increased or decreased by incrementing the motor either clockwise or counterclockwise. This in turn will rotate the compression insert, which will raise or lower via its external threading.

Motor design can use a stepper motor, brushed DC, or brushless DC. Depending on the accuracy required a rotary encoder can be incorporated, being placed either on the output shaft of the spring preload motor or on the spring compression gear face.

Impact Energy Control Mechanism, Friction

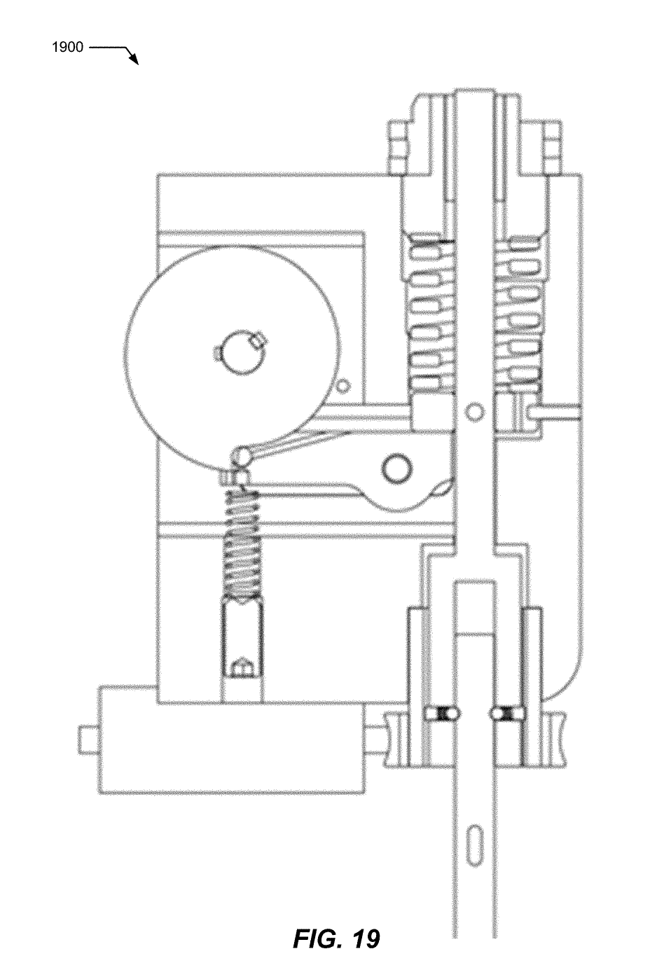

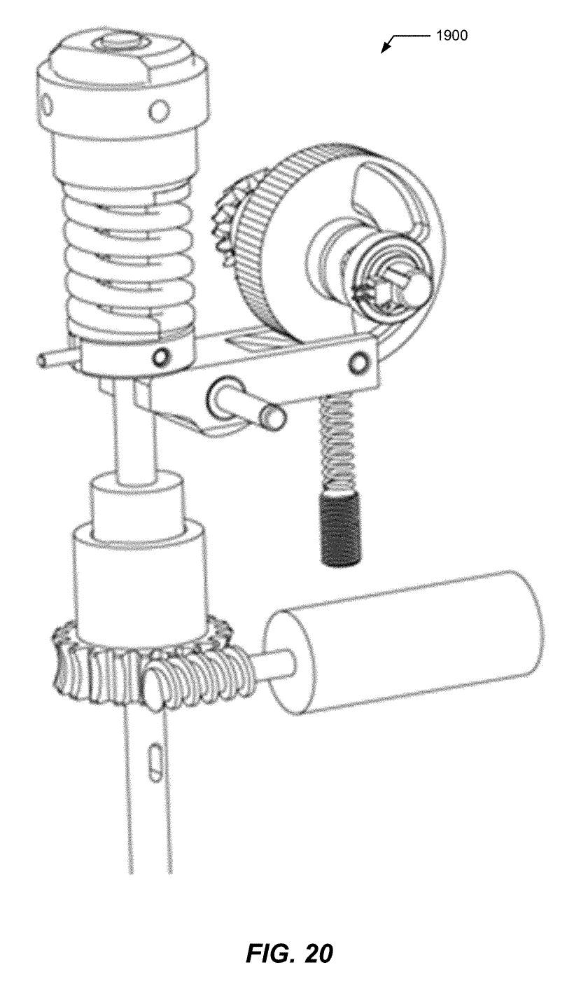

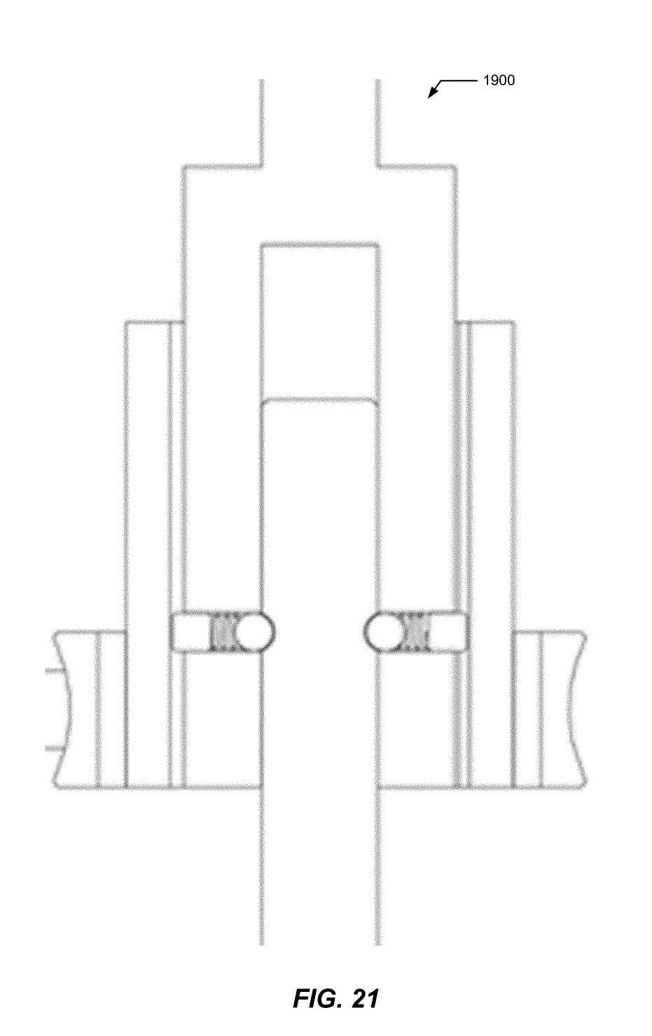

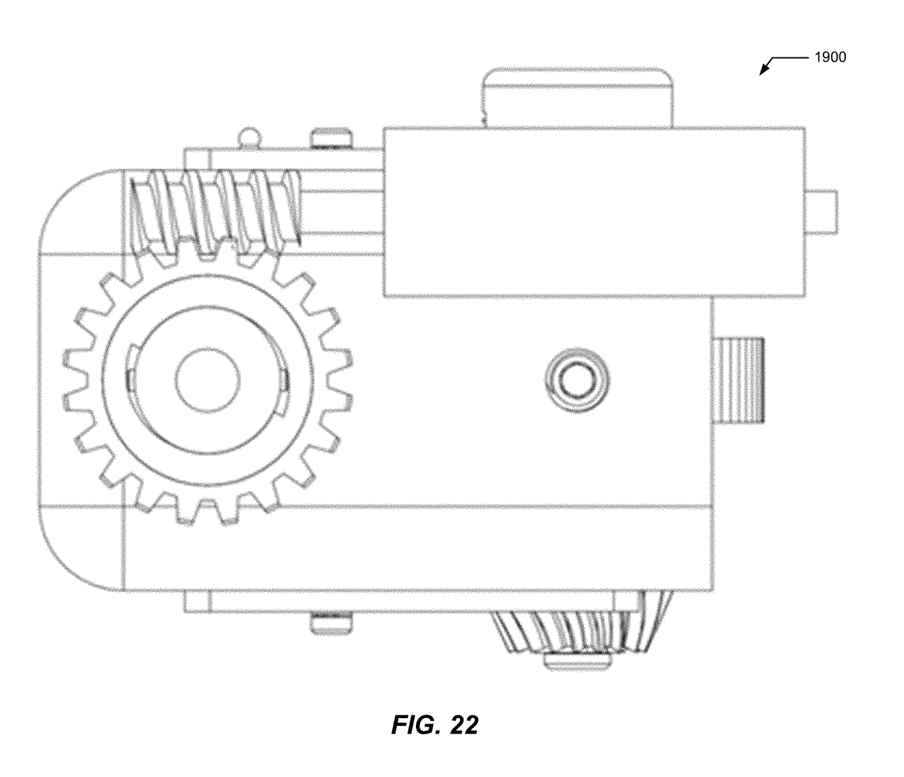

FIG. 19 illustrates cross-sectional view of an impact energy control mechanism (friction) 1900 as may be used in the particular BMD of FIG. 14; FIG. 20 illustrates an internal view of an impact energy control mechanism (friction) 1900 as may be used in the particular BMD of FIG. 14; FIG. 21 illustrates a close-up detail of an impact energy control mechanism (friction) 1900, ball-detent as may be used in the particular BMD of FIG. 14; and FIG. 22 illustrates a bottom view of an impact energy control mechanism (friction) 1900 as may be used in the particular BMD of FIG. 14.

The second example would have a static spring preload, and would instead us friction to control the amount of energy transferred for each impact. The shaft spring would strike a hollow tube, which would fit over a distal instrument shaft. One or more ball plungers would be threaded through the wall of the tube, pressing onto the side of the instrument shaft. The insertion depth of the ball plungers could be controlled via a motor and ball detent control gear, which in turn would determine the friction forces between the tube and the instrument shaft. The ball detent control gear would have a cam inner profile, allowing the depth of the ball plungers to be varied depending on the rotational position of the gear. The friction force generated by the ball plungers would determine the amount of energy that would be transmitted to the instrument shaft, with any excess spring forces resulting in slip between the tube and shaft.

Acetabulum Cup Insertion Device, Slide Hammer, Hand Operated Concept

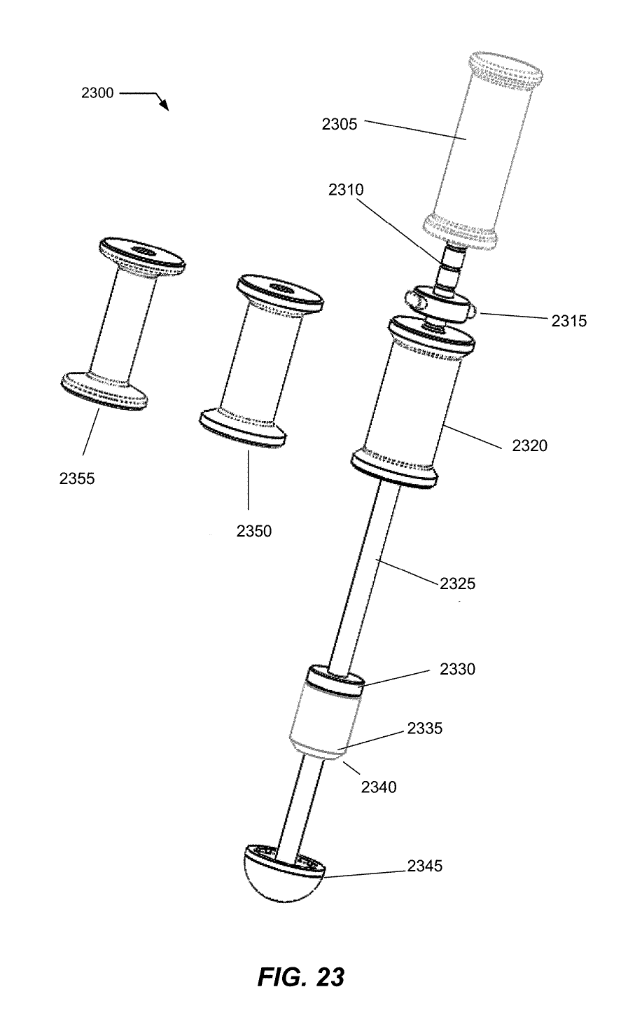

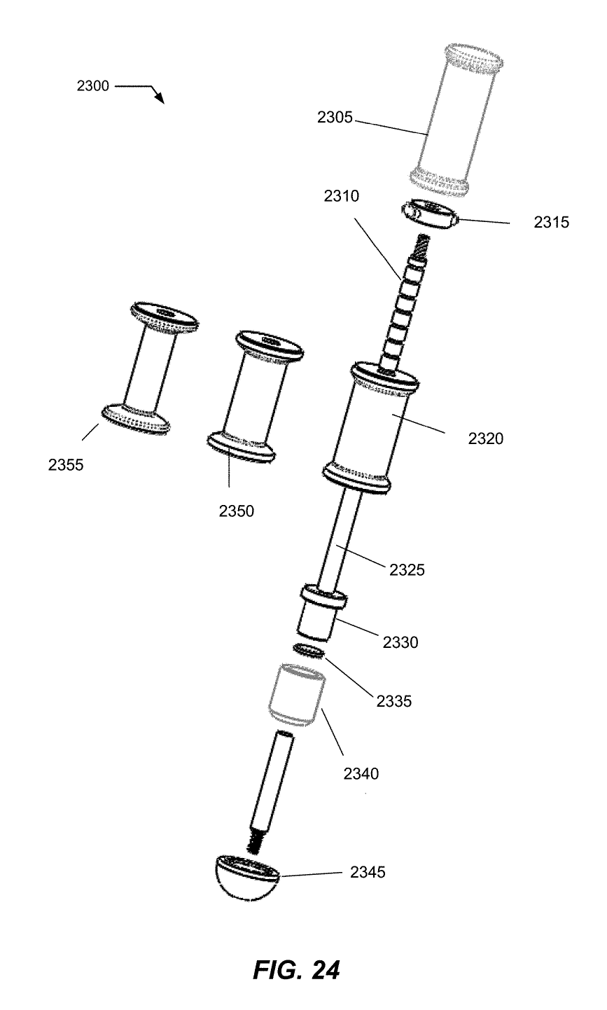

FIG. 23-FIG. 24 relate to a second particular implementation of a mechanical BMD for controlled axial impact. FIG. 23 illustrates a hand-operated slide hammer implementation for a mechanical BMD 2300; and FIG. 24 illustrates an exploded view of mechanical BMD 2300. BMD 2300 includes a fixed grip 2305, a set of travel stop adjustment grooves 2310, a slide travel stop adjuster 2315, a heavy slide 2320, a slide shaft 2325, a force sensor top 2330, a force sensor 2335, a force sensor bottom 2340, an acetabular cup 2345, a medium slide 2350, and a light slide 2355 (slides represent variable mass for varying force).

Acetabulum cup insertion involves striking an insertion shaft threaded to the replacement cup with a free-swinging hammer to seat the cup. Alignment and full seating of the cup is a trial-and-error process, involving much corrective striking of the insertion shaft to properly seat the cup. The many variables involved in this process include striking force, direction of strike on the insertion shaft, and hammer weight. If done incorrectly, damage to the patient may result. The slide-hammer insertion device is designed to minimize these liabilities by making each force input separate from the others, and by helping to constrain each force input to a controlled factor.

Each force input is separated into a controllable vector: Direction - The Slide Shaft directs the seating force of each impact. Impact Mass: The Impact Sliders come in a range of rates. The heavier they are, the greater the impact force, and the greater the “dwell time”, or duration of the impact. Slide Distance: The Slide Travel Adjuster limits the acceleration and therefore the impact speed of the hammer weight. Impact Force Sensor: Indicates the force generated with each impact, giving the surgeon a comparison to the optimal desired impact for each combination of cup size and type and bone density.

Resultant Forces:

Combinations of the slide weights and the travel distance can be tabulated to take into ac-count the surgeon's strength, the patient's bone density, and the size and type of Acetabular Cup being used. Insertion direction can be adjusted between each impact to reduce the amount of corrective impact needed to properly seat the cup. A navigation system may be employed to assist in proper orientation during installation of cup 2345.

Acetabulum Cup Insertion Device, Slide Hammer, Pneumatic Concept

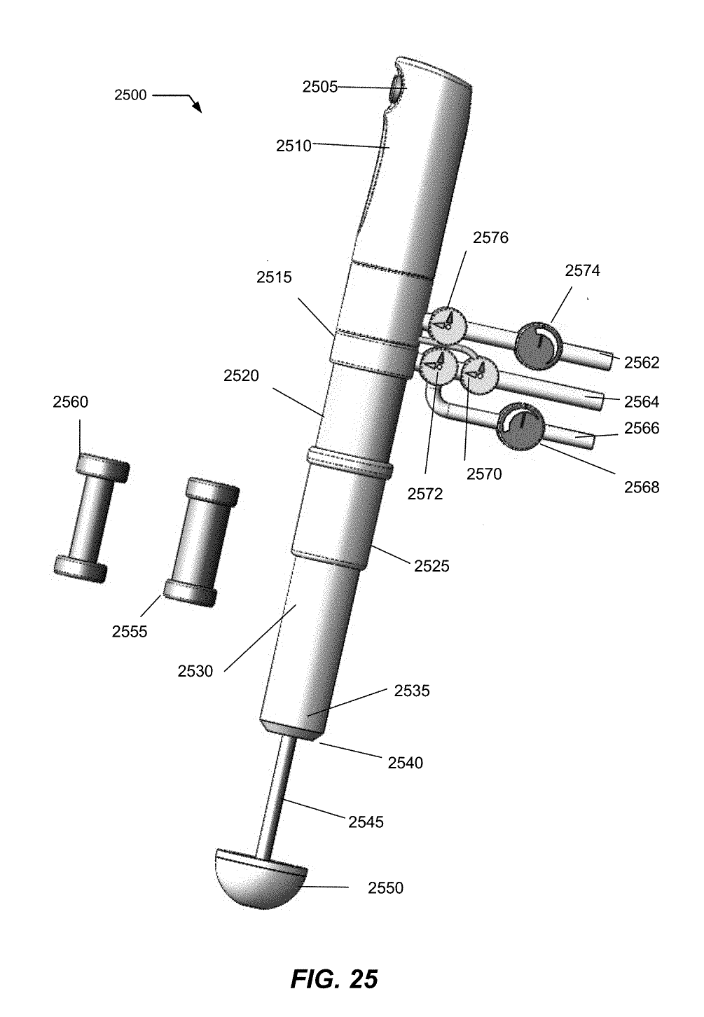

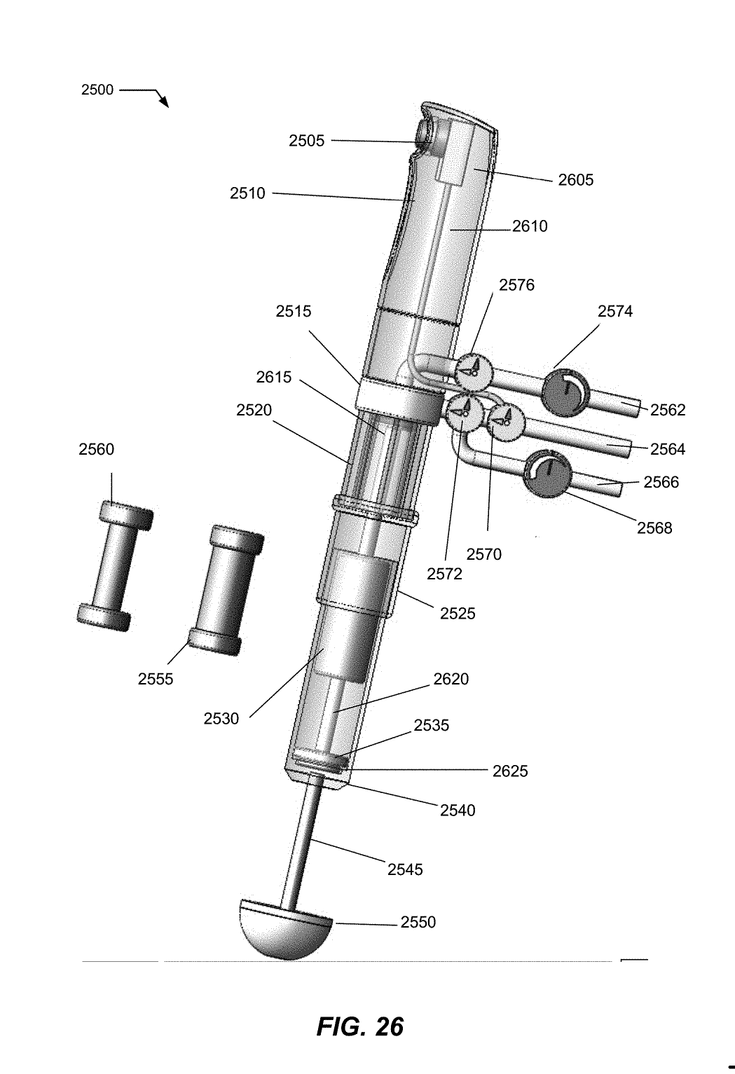

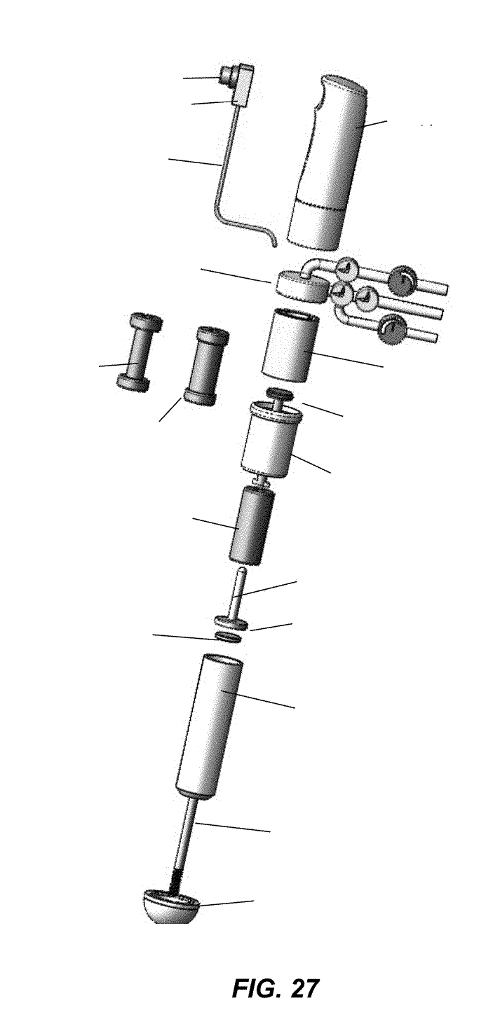

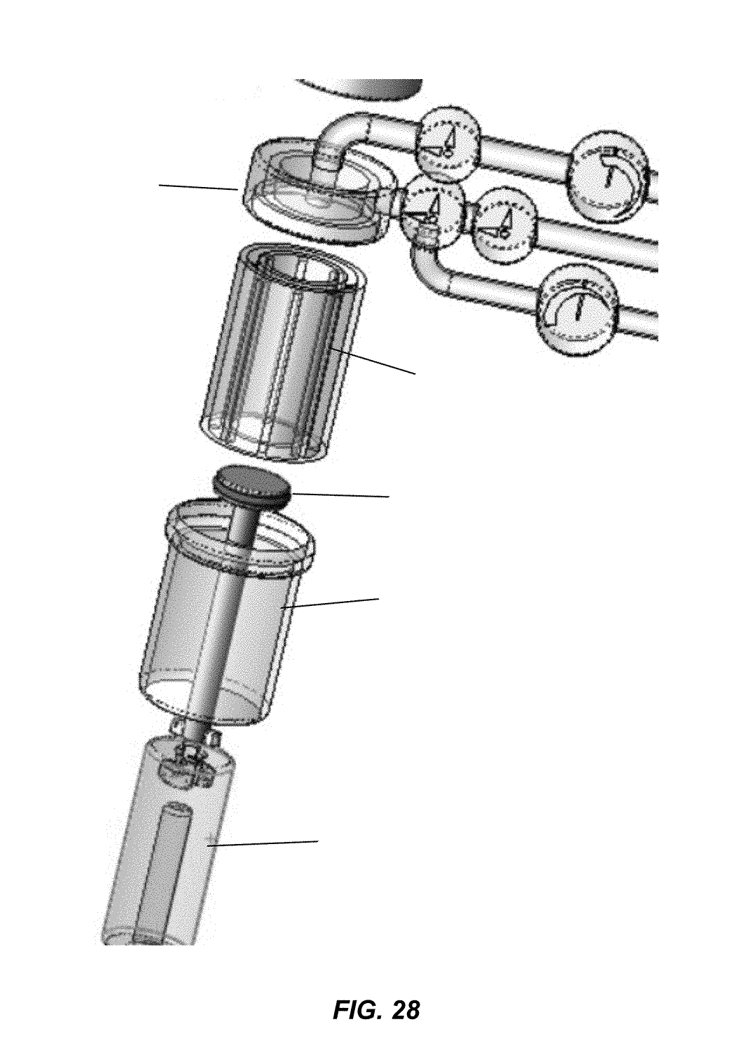

FIG. 25-FIG. 27 relate to a third particular implementation of a mechanical BMD for controlled axial impact; FIG. 25 illustrates a pneumatically-operated slide hammer implementation for a mechanical BMD 2500; FIG. 26 illustrates an internal view of the mechanical BMD of FIG. 25; FIG. 27 illustrates an exploded view of the mechanical BMD of FIG. 25; and FIG. 28 illustrates a detail view of the pneumatic engine for the BMD of FIG. 25. BMD 2500 includes a trigger 2505, an upper grip 2510, an air manifold 2515, a cylinder 2520, a travel adjust tube 2525, a heavy slide 2530 (inside), an impact plate 2535, a slide tube/lower grip 2540, a cup shaft 2545, a cup 2550, a medium slide 2555, ad light slide 2560 (slides interchangeable with heavy slide 2530). Pneumatic system further includes an air input 2562, an air exhaust 2564, a reset air input 2566, a reset actuation pressure control 2568, an exhaust valve 2570, a reset valve 2572, a piston actuation pressure control 2574, and a piston actuation valve 2576. Further illustrated in FIG. 26, BMD 2500 includes air actuator control circuits 2605, air actuator wiring 2610, piston and rod 2615, a slide guide 2620, and a force sensor 2625.

The current state-of-the-art acetabulum cup insertion involves striking an insertion shaft threaded to the replacement cup with a free-swinging hammer to seat the cup. Alignment and full seating of the cup is a trial-and-error process, involving much corrective striking of the insertion shaft to properly seat the cup. The many variables involved in this process include striking force, direction of strike on the insertion shaft, and hammer weight. If done incorrectly, damage to the patient may result.

The slide-hammer insertion device is designed to minimize these liabilities by making each force input separate from the others, and by helping to constrain each force input to a controlled factor. Additionally, this concept uses air to move the impact weight, making the application of force more predictable across a range of users, regardless of strength and size.

Each force input is separated into a controllable vector: Direction: The Slide Shaft directs the seating force of each impact. Impact Mass: The Impact Sliders come in a range of rates. The heavier they are, the greater the impact force, and the greater the “dwell time”, or duration of the impact. Pneumatic force: is adjustable to take the user variability out of the equation. Impact Force Sensor: Indicates the force generated with each impact, giving the surgeon a comparison to the optimal desired impact for each combination of cup size and type and bone density.

Resultant Forces:

Combinations of the slide weights and the air pressure can be tabulated to take into account the patient's bone density and the size and type of Acetabular Cup being used. Insertion direction can be adjusted between each impact to reduce the amount of corrective impact needed to properly seat the cup.

The system and methods above has been described in general terms as an aid to understanding details of preferred embodiments of the present invention. In the description herein, numerous specific details are provided, such as examples of components and/or methods, to provide a thorough understanding of embodiments of the present invention. Some features and benefits of the present invention are realized in such modes and are not required in every case. One skilled in the relevant art will recognize, however, that an embodiment of the invention can be practiced without one or more of the specific details, or with other apparatus, systems, assemblies, methods, components, materials, parts, and/or the like. In other instances, well-known structures, materials, or operations are not specifically shown or described in detail to avoid obscuring aspects of embodiments of the present invention.

Reference throughout this specification to “one embodiment”, “an embodiment”, or “a specific embodiment” means that a particular feature, structure, or characteristic described in connection with the embodiment is included in at least one embodiment of the present invention and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment of the present invention may be combined in any suitable manner with one or more other embodiments. It is to be understood that other variations and modifications of the embodiments of the present invention described and illustrated herein are possible in light of the teachings herein and are to be considered as part of the spirit and scope of the present invention.

It will also be appreciated that one or more of the elements depicted in the drawings/figures can also be implemented in a more separated or integrated manner, or even removed or rendered as inoperable in certain cases, as is useful in accordance with a particular application.

Additionally, any signal arrows in the drawings/Figures should be considered only as exemplary, and not limiting, unless otherwise specifically noted. Combinations of components or steps will also be considered as being noted, where terminology is foreseen as rendering the ability to separate or combine is unclear.

The foregoing description of illustrated embodiments of the present invention, including what is described in the Abstract, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of illustrated embodiments of the present invention and are to be included within the spirit and scope of the present invention.

Thus, while the present invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the present invention. It is intended that the invention not be limited to the particular terms used in following claims and/or to the particular embodiment disclosed as the best mode contemplated for carrying out this invention, but that the invention will include any and all embodiments and equivalents falling within the scope of the appended claims. Thus, the scope of the invention is to be determined solely by the appended claims.

1-33. (canceled)

34: An axially-impactful device for imparting a force to a prosthesis to be installed in an installation direction with the prosthesis including an attachment structure, comprising:
a rod having a shaft including a proximal end, a distal end spaced apart from said distal end, and a longitudinal axis extending from said proximal end to said distal end through said rod, said distal end including an engagement structure complementary to the attachment structure;
an impact energy control coupled to said proximal end;
a rocker assembly including a proximal end and a distal end, spaced away from said proximal end and coupled to said impact energy control;
a cam including a cam surface coupled to said proximal end of said rocker assembly;
an engine producing a rotary engine motion; and
a gear train coupled to said engine and to said cam with said gear train configured to convert said rotary engine motion to a rotary cam motion of said cam surface.

35: The device of claim 34 wherein said cam surface includes a cam profile and wherein said impact energy control is responsive to said cam profile, and wherein said impact energy control periodically stores and releases energy to said shaft and is configured to produce a series of discrete impacts aligned with said longitudinal axis.

36: The device of claim 35 wherein each said discrete impacts has a predetermined minimum impact force.

37: The device of claim 36 further comprising a variable control coupled to said impact energy control to set a desired variable value for said predetermined minimum impact force.

38: The device of claim 34 wherein said impact energy control includes a spring preload assembly.

39: The device of claim 34 wherein said impact energy control includes a friction-controlled assembly.

40: The device of claim 35 wherein said impact energy control includes a spring preload assembly.

41: The device of claim 35 wherein said impact energy control includes a friction-controlled assembly.

42: The device of claim 36 wherein said impact energy control includes a spring preload assembly.

43: The device of claim 36 wherein said impact energy control includes a friction-controlled assembly.

44: The device of claim 37 wherein said impact energy control includes a spring preload assembly.

45: The device of claim 37 wherein said impact energy control includes a friction-controlled assembly.

46: A method for imparting a force to a prosthesis to be installed in an installation direction with the prosthesis including an attachment structure, comprising:
a) rocking cyclically a first end of a first arm of a rocker assembly;
b) compressing cyclically an impact energy control coupled to said first arm responsive to said rocking step a);
c) releasing cyclically said impact energy control responsive to said rocking step c) to produce a series of discrete impacts; and
d) coupling said series of discrete impacts to the prosthesis through a shaft coupled to said impact energy control and to the prosthesis with said shaft having a longitudinal axis aligned with the installation direction; and
wherein said series of discrete impacts are aligned with said longitudinal axis.

47: An axially-impactful device for imparting a force to a prosthesis to be installed in an installation direction with said prosthesis including an attachment structure, comprising:
a pneumatic engine producing a controllable air flow;
a rod having a shaft including a proximal end, a distal end spaced apart from said proximal end, an impact plate at said distal end, and a longitudinal axis extending from said proximal end to said distal end through said rod;
a slide, responsive to said controllable air flow, slidingly coupled to said shaft to deliver a series of discrete axial impacts against said impact plate; and
an attachment system coupled to said distal end, said attachment system configured to both engage the attachment structure and align said longitudinal axis with the installation direction.

48: The device of claim 48 further comprising a force sensor coupled to said impact plate.
